(19) United States
(12) Patent Application Publication
(10) Pub. No.: US 2025/0144020 A1
(43) Pub. Date: May 8, 2025

(54) ULTRA-LONG FLOATING HYDROGEL RAFT FOR PROLONGED GASTRIC RETENTION AND APPLICATIONS REQUIRING BUOYANCY WITH CONTROLLED-RELEASE

(71) Applicant: Nanyang Technological University, Singapore (SG)

(72) Inventors: Say Chye Joachim LOO, Singapore (SG); Kaarunya SAMPATHKUMAR, Singapore (SG); Guo Dong KWANG, Singapore (SG)

(21) Appl. No.: 18/837,816

(22) PCT Filed: Feb. 14, 2023
(86) PCT No.: PCT/SG2023/050080

§ 371 Date: Aug. 12, 2024

(30) Foreign Application Priority Data
Feb. 18, 2022 (SG) 10202201599S

(51) Int. Cl. A61K 9/06; A61K 9/00; A61K 47/02; A61K 47/36; A61K 47/44

(52) U.S. Cl. CPC A61K 9/06; A61K 9/0065; A61K 47/02; A61K 47/36; A61K 47/44

(57) ABSTRACT

Disclosed herein are a pharmaceutical flotation device suitable for extended release of a pharmaceutical product in a stomach of a subject, the flotation device comprising a polymeric matrix that comprises a first polymeric material, a second polymeric material, and a crosslinking agent, wherein the first polymeric material and the second polymeric material form a hydrogel network; and the crosslinking agent is configured to generate additional crosslinks in at least the first polymeric material upon exposure to an aqueous solution having a pH of from 1 to 5, and a method of forming the pharmaceutical flotation device.

### FIELD OF INVENTION

This invention relates to a method of preparing an orally administrable depot for controlled and sustained release of encapsulated agents. In particular, this invention relates to the preparation of an ultralong floating raft that is designed to entrap drugs or drug loaded devices that can be resident in the stomach. With this delivery system, the aim is to reduce dosing frequency and pill burden, thus improving patient medication compliance in chronic disease conditions especially in the elderly.

### BACKGROUND

The listing or discussion of a prior-published document in this specification should not necessarily be taken as an acknowledgement that the document is part of the state of the art or is common general knowledge.

Medication non-adherence is a persistent problem that jeopardises the effectiveness of healthcare systems. Poor adherence, observed nearly in 50-60% of patients, is the primary reason for not achieving the full health benefits medicines can provide. It causes medical and psychosocial complications of disease, reduces patients' quality of life, increases the likelihood of development of drug resistance, and underutilizes health care resources. The problem is further compounded in the case of polypharmacy prescription in elderly when some of the chronic conditions predispose the development of another chronic condition, with non-adherence accounting for nearly 50% treatment failures.

The rapid advances and accessibility in healthcare have drastically improved the average life expectancy. Based on a study in 2020, approximately 65% of those aged 65 years or more and 82% of those aged 85 years or more have two or more chronic conditions (Maidment, I., Huckerby, C. & Shukla, D., Prescriber 2020, 31, 30-33). However, findings have shown that approximately 1 in 4 adults suffers from 1 chronic condition or multiple chronic conditions (Marengoni, A. et al., Ageing Res. Rev. 2011, 10, 430-439), with the figure rising to 3 out of 4 older adults in developed countries and is expected to rise exponentially (Buttorff, C., Ruder, T. & Bauman, M., Multiple chronic conditions in the United States. Rand Santa Monica, CA: 2017, Vol. 10). In terms of mortality, chronic diseases account for 41 million global deaths annually, constituting approximately 71% of annual global death rates.

The top five chronic health conditions in elderly are: high blood pressure; high blood cholesterol; neurodegenerative diseases; joint pain, arthritis, rheumatism, or nerve pain; and diabetes, all of them requiring long-term medication to manage the conditions. Poor adherence to the long-term treatment of chronic diseases is an increasing, world-wide problem of striking magnitude.

Gastro resident drug delivery systems (GRDDS), exploit the use of stomach as a drug depot, offering to increase residence time of the drug in the body, by controlling the release kinetics of the drug compared to a readily dissolving tablet dosage form. GRDDS function by different mechanisms such as floating-effervescence, low density, porosity, hollow cavity and non-floating-mucoadhesive, high density, and raft forming systems. There have been a few GRDDS that have also been approved for commercial use (Vrettos, N.-N., Roberts, C. J. & Zhu, Z., Pharmaceutics 2021, 13, 1591; and Tripathi, J. et al., Pharmaceutics 2019, 11, 193). However, most of the formulations reported so far have a maximum floatation time of 24 h with gas generation being the major mechanism of floatation.

Previously reported floating delivery systems are mostly based on effervescence and they supported floatation up to a maximum time of 24 hours, depending on the gas generation reagent (usually carbonates) within them. In the case of noneffervescent GRDDS, the delivery devices utilise mucoadhesive properties or gastric obstruction designs. However, mucoadhesive-based devices are removed when the mucus layer in the upper gastric tract is shed and replenished, as seen in most chitosan-based polymeric devices. On the other hand, devices based on gastric obstruction designs may lead to problems in gastric emptying. One such gastric obstruction device is Lyndra's™ technology that has incorporated automatic degradation of the structure after the intended time. Still, the design is heavily dependent on polymers produced via melt and heat extrusion. This places a limitation on the type of drugs for encapsulation due to various thermal degradation or chemical interaction exposed to them.

Conventional floating drug delivery systems involve systems whereby only free drugs can be encapsulated, and most often lack controlled release capabilities.

Therefore, there exists a need for a new floatable GRDSS that overcomes the above-mentioned problems of pill burden and poor adherence to medication in chronic diseases, to prolong the floatability of the system, thereby providing ultralong release of drugs from the delivery system.

### SUMMARY OF INVENTION

Aspects and embodiments of the invention will now be described by reference to the following numbered clauses.

1. A pharmaceutical flotation device suitable for extended release of a pharmaceutical product in a stomach of a subject, the flotation device comprising a polymeric matrix that comprises:
a first polymeric material;
a second polymeric material; and
a crosslinking agent, wherein:
   the first polymeric material and the second polymeric material form a hydrogel network; and the crosslinking agent is configured to generate additional crosslinks in at least the first polymeric material upon exposure to an aqueous solution having a pH of from 1 to 5.

2. The pharmaceutical flotation device according to Clause 1, wherein the pharmaceutical flotation device is provided in a first portion shaped as a hollow vessel and one or more second portions shaped as a cap and/or a plug, such that a pharmaceutical product can be deposited within the hollow vessel and sealed inside by the cap and/or plug.

3. The pharmaceutical flotation device according to Clause 1, wherein a pharmaceutical product is encapsulated within the polymeric matrix of the pharmaceutical flotation device.

4. The pharmaceutical flotation device according to any one of the preceding clauses, wherein the polymeric matrix further comprises an oil, optionally wherein the oil is selected from one or more of the group consisting of olive oil, fish oil, corn oil, sunflower seed oil, grape seed oil, canola oil, avocado oil, and coconut oil (e.g. the oil is coconut oil).

5. The pharmaceutical flotation device according to any one of the preceding clauses, wherein the first polymeric material is selected from one or more of the group consisting of a pectin, and an alginate.

6. The pharmaceutical flotation device according to any one of the preceding clauses, wherein the second polymeric material is selected from one or more of the group consisting of resistant starch, kappa-carrageenan, agarose, iota-carrageenan, cellulose, methylcellulose, ethyl cellulose, and gelatine.

7. The pharmaceutical flotation device according to Clause 6, wherein the second polymeric material is kappa-carrageenan.

8. The pharmaceutical flotation device according to any one of the preceding clauses, wherein the crosslinking agent is a bivalent metal carbonate.

9. The pharmaceutical flotation device according to Clause 8, wherein the bivalent metal carbonate is CaCO₃.

10. The pharmaceutical flotation device according to any one of the preceding clauses, wherein the wt:wt ratio of the first polymeric material to the second polymeric material is from 1:10 to 10:1, such as about 1:1.

11. The pharmaceutical flotation device according to any one of the preceding clauses, wherein the wt:wt ratio of the crosslinking agent to the first polymeric material is from 1:10 to 10:1, such as about 1:1.

12. The pharmaceutical flotation device according to any one of the preceding clauses, wherein the polymeric matrix comprises:
sodium alginate;
kappa-carageenan; and
CaCO₃, where
   the wt:wt ratio of the sodium alginate:kappa-carageenan:CaCO₃ is about 1:1:1, optionally wherein the polymeric matrix further comprises coconut oil.

13. The pharmaceutical flotation device according to any one of Clauses 2 and 4 to 12, as dependent upon Clause 2, wherein the pharmaceutical flotation device further comprises a pharmaceutical product.

14. The pharmaceutical flotation device according to any one of Clauses 3, 4 to 12 as dependent upon Clause 3, and Clause 13, wherein the pharmaceutical product is in the form of: particles of one or more active pharmaceutical ingredients; particles of one or more active pharmaceutical ingredients and pharmaceutically acceptable excipients; a tableted formulation; a capsule formulation; drug-loaded microparticles; and drug-loaded fibre meshes.

15. The pharmaceutical flotation device according to any one of the preceding clauses, wherein the hydrogel network is an interpenetrating network.

16. A method of forming a pharmaceutical flotation device according to any one of Clauses 1 to 15, the method comprising the steps of:
(a) providing a solution mixture comprising:
   a first polymeric material;
   a second polymeric material;
   a crosslinking agent; and
   water, where the first polymeric material and the second polymeric material interact to form a hydrogel network;
(b) placing the solution mixture into a mould, solidifying the solution mixture and removing the water to provide a pharmaceutical flotation device.

17. The method according to Clause 16, wherein step (b) is conducted using at least two moulds, so as to form a first portion shaped as a hollow vessel and one or more second portions shaped as a cap and/or a plug, such that a pharmaceutical product can be deposited within the hollow vessel and sealed inside by the cap and/or plug.

18. The method according to Clause 16 or Clause 17, wherein the solution mixture further comprises a pharmaceutical product.

19. The method according to any one of Clauses 16 to 18, wherein the solution mixture further comprises an oil, optionally wherein the oil is selected from one or more of the group consisting of olive oil, fish oil, corn oil, sunflower seed oil, grape seed oil, canola oil, avocado oil, and coconut oil (e.g. the oil is coconut oil).

20. The method according to any one of Clauses 16 to 19, wherein the first polymeric material is selected from one or more of the group consisting of a pectin, and an alginate.

21. The method according to any one of Clauses 16 to 20, wherein the second polymeric material is selected from one or more of the group consisting of resistant starch, kappa-carrageenan, agarose, iota-carrageenan, cellulose, methylcellulose, ethyl cellulose, and gelatine, optionally wherein the second polymeric material is kappa-carrageenan.

22. The method according to any one of Clauses 16 to 21, wherein the solution mixture of step (a) is heated to a temperature of greater than 40° C. for a first period of time and is then cooled to about 40° C. for a second period of time before step (b) of the method is conducted, optionally wherein the mould in step (b) has been cooled to a temperature of less than 25° C.

23. The method according to any one of Clauses 16 to 22, wherein the crosslinking agent is a bivalent metal carbonate.

24. The method according to Clause 23, wherein the bivalent metal carbonate is CaCO₃.

25. The method according to any one of Clauses 16 to 24, wherein the wt:wt ratio of the first polymeric material to the second polymeric material is from 1:10 to 10:1, such as about 1:1.

26. The method according to Clause 25, wherein the maximum concentration of each of the first polymeric material and the second polymeric material in the solution mixture is from 1 to 2 wt %/volume, such as about 1.5 wt %/volume.

27. The method according to any one of Clauses 16 to 26, wherein the wt:wt ratio of the crosslinking agent to the first polymeric material is from 1:10 to 10:1, such as about 1:1.

28. The method according to Clause 27, wherein the maximum concentration of crosslinking agent in the solution mixture is from 1 to 2 wt %/volume, such as about 1.5 wt %/volume.

29. The method according to any one of Clauses 18 and 19 to 28, as dependent upon Clause 18, wherein the pharmaceutical product is in the form of: particles of one or more active pharmaceutical ingredients; particles of one or more active pharmaceutical ingredients and pharmaceutically acceptable excipients; a tableted formulation; a capsule formulation; drug-loaded microparticles; and drug-loaded fibre meshes.

30. The method according to any one of Clauses 16 to 29, wherein the hydrogel network is an interpenetrating network.

31. A pharmaceutical formulation comprising a pharmaceutical flotation device according to any one of Clauses 1 to 15 and a pharmaceutical product.

32. The pharmaceutical formulation according to Clause 31, wherein the pharmaceutical product is in the form of: particles of one or more active pharmaceutical ingredients; particles of one of more active pharmaceutical ingredients and pharmaceutically acceptable excipients; a tableted formulation; a capsule formulation; drug-loaded microparticles; and drug-loaded fibre meshes.

### DRAWINGS

FIG. 1 depicts photographic images of the various moulds used to fabricate the rafts. (a) Hard mould for round tablet. (b) Separable mould with 2 components, attached (left) and detached hard and silicon components. (c) Single soft silicon mould. (d) Plastic mould for receptacle cap. (e) Makeshift mould using 24 well-plate and cap for receptacle base. (f) Freeze-dried sample of receptacle base removed from well-plate. (g) Mould setup used to fabricate the base of the receptacle cap assembly.

FIG. 2 depicts (a) schematic showing the fabrication process of the raft, photographs showing the (b) Kappa Carrageenan (KC)-Sodium Alginate (Alg) raft fabricated from the tablet-shaped mould and (c) KC-Alg raft fabricated using the receptacle cup type mould, and scanning electron micrographs (SEM) of the (d) planar cross-sectional view and (e) vertical cross-sectional view of the KC-Alg raft to show the surface and internal morphology, respectively. All samples were incubated at 37° C. and shaken at 150 rpm.

FIG. 3 depicts photographic images of the KC-Alg (CaCO₃) rafts in fasted state simulated gastric fluid (FaSGF) with enzymes taken (a) after 24 h in the medium, (b) after 14 days in the medium, and (c) after subjecting the gels to multiple cycles of pH switch between FaSGF and fed state simulated gastric fluid (FeSGF) for seven days. All samples were incubated at 37° C. and shaken at 150 rpm.

FIG. 4 depicts the photographic images of the KC-Alg (mix) raft in FaSGF with enzymes taken (a) after 24 h in the medium (b) after 14 days in the medium, and (c) after subjecting the gels to four cycles of pH switch between fasted and fed state SGF. All samples were incubated at 37° C. and shaken at 150 rpm.

FIG. 5 depicts the scanning electron micrographs of (A) vertical cross-section of KC-Alg (mix), and (B) planar cross-section of KC-Alg (mix).

FIG. 6 depicts the photographs showing the (a) KC-Alg raft and (b) KC-Alg raft floating in 35 simulated gastric fluid (SGF) after 24 days. Scanning electron micrographs of the (c) top view and (d) cross-sectional view of the KC-Alg raft to show the surface and internal morphology, respectively. All samples were incubated at 37° C. and shaken at 150 rpm.

FIG. 7 depicts the compression test of KC-Alg raft using a 50 N load at a speed of 10 mm/min under 50% strain. The raft was subject to pH switch over a seven-day period. Each raft was subject to four cycles of switching between FaSGF and FeSGF (lasting 1.5 h for one switch) on each day, before being subject to compression testing. After the compression test, the sample was tested for its buoyancy.

FIG. 8 depicts photographic images showing the triggered degradation of KC-Alg raft upon the addition of trisodium citrate at different concentrations: (a) 0.2 M; (b) 0.1 M; (c) 0.05 M; and (d) 0.025 M, indicating a concentration dependent effect.

FIG. 9 depicts the scanning electron micrographs showing the (a) microparticles (MPs) and their (b) cross-sectional morphology.

FIG. 10 depicts (a) photograph of the fiber mesh (FM) obtained by peeling off the electrospun fibers from aluminium foil, and (b and c) scanning electron micrographs of the FM at different magnifications ((b): 500× (scale bar is 20 um), and (c): 4000× (scale bar is 5 um)).

FIG. 11 depicts the release profiles of metoprolol tartrate (MT) from (a) free MT encased in the raft, (b) MPs encapsulated with MT encased in the raft, and (c) FM encapsulated with MT encased in the raft. Each group was compared against the same form of the drug as a control, without encasing in the raft. Release study was done in FaSGF at 37° C. in a shaking incubator at 150 rpm.

FIG. 12 depicts graphs showing the modelling of MT release data from (a) raft (FD-R), (b) fibers free (FM) and encased in raft (FM-R), and (c) MPs free (MP) and encased in raft (MP-R).

FIG. 13 depicts the release profiles of Risperidone (Ris) from (a) free Ris encased in the raft, (b) commercial Ris tablet encased in raft, (c) MPs encapsulated with Ris encased in the raft, and (d) FM encapsulated with Ris encased in the raft. Each group was compared against the same form of the drug as a control, without encasing in the raft. Release study was done in FaSGF at 37° C. in a shaking incubator at 150 rpm.

FIG. 14 depicts graphs showing the modelling of Ris release data from (a) raft (FD-R), (b) fibers free (FM) and encased in raft (FM-R), and (c) MPs free (MP) and encased in raft (MP-R).

FIG. 15 depicts the comparison of the release rate kinetics of MT and Ris from the different formulation of the drugs based on the Higuchi equation.

FIG. 16 depicts the graph for the empirical formula to calculate k_{H} based on the log P values of the drugs when encapsulated in the MP-R formulation.

### DESCRIPTION

Some or all of the problems discussed above are solved by the currently claimed invention. For example, the present invention provides a GRDDS that can remain floating under gastric conditions for up to a month without any lag time in floating.

The currently disclosed invention not only provides a pharmaceutical flotation device (e.g. a dry hydrogel raft) capable of prolonged floatation within the gastric environment, but also uses a mild fabrication technique that can be applied to a variety of drugs. Three different floating mechanisms may be incorporated into the pharmaceutical flotation devices disclosed herein to ensure prolonged floatation. In the first instance, a dual gel network between a first and a second polymeric material (e.g. an alginate and kappa-carrageenan) produces a highly integrated porous gel network aiding in both drug release and floatability. For example, when the second polymeric material is kappa-carrageenan, heat treatment and subsequent cooling of the kappa-carrageenan can result in a phase transition of the kappa-carrageenan to strengthen the overall hydrogel network and the low working temperature allows for greater selection of drugs to be added into the raft in the wet state (i.e. during production of the desired pharmaceutical flotation device). The addition of an oil (e.g. coconut oil) may enhance floatation of the pharmaceutical flotation device (also referred to herein as a hydrogel raft, or raft for short) and this may also allow for the incorporation of hydrophobic drugs in the otherwise hydrophilic gel network. The first and second polymeric materials (e.g. alginate and kappa-carrageenan) may display affinity for ionic crosslinking (e.g. via CaCO₃). As such, a crosslinking agent that is capable of releasing a bivalent (or higher) metal ion may be included in the pharmaceutical flotation device. This crosslinking agent may be relatively insoluble in water, but may then release the bivalent metal ion to provide further crosslinking of at least the first polymeric material (and, in some cases, the second polymeric material too) upon exposure to an acidic environment (e.g. stomach acid). Given this, the crosslinking agent may exhibit a synergistic mechanical strengthening effect when ingested in a fasted state (thereby ensuring the lowest-possible pH in the stomach). The present invention also provides the option of fabricating a drug-encapsulated raft (formed in the wet state) as well as a dry receptacle design. This allows for easy adoption of the device with any drugs or even other delivery systems, enabling a wider translation range from bench to bedside application.

The pharmaceutical flotation device disclosed herein may use natural ingredients and polymers that are safe for consumption. For example, the first and second polymeric material (e.g. alginate and kappa-carrageenan) may be classified as generally recognised as safe (GRAS). Such polymers may frequently be used as food thickeners, which lends greater consumer confidence that the device disclosed here is safe for consumption.

In addition, the fabrication process is simple which allows for easy scale-up for high throughput production.

The present invention can also offer greater versatility in controlling drug release rates by manipulating different parameters, i.e., type and ratio of the hydrogels used. Unlike other methods of producing floatable delivery systems, high temperature and compression forces are not required in this technique. On the other hand, only simple and economical apparatus are required, that can be easily scaled up for high throughput fabrication.

In a first aspect of the invention, there is provided a pharmaceutical flotation device suitable for extended release of a pharmaceutical product in a stomach of a subject, the flotation device comprising a polymeric matrix that comprises:
a first polymeric material;
a second polymeric material; and
a crosslinking agent, wherein:
   the first polymeric material and the second polymeric material form a hydrogel network; and the crosslinking agent is configured to generate additional crosslinks in at least the first polymeric material upon exposure to an aqueous solution having a pH of from 1 to 5.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

When used herein, the “first polymeric material” is a material that can undergo crosslinking when exposed to a suitable crosslinking agent under the correct conditions. In addition, the first polymeric material should be a material that is capable of forming a hydrogel network with the second polymeric material.

The first polymeric material may be any suitable polymeric material with the properties discussed above. In embodiments that may be mentioned herein, the first polymeric material may be a pectin, an alginate or combinations thereof. In particular embodiments that may be mentioned herein, the first polymeric material may be an alginate.

When used herein, the “second polymeric material” should be a material that is capable of at least some degree of self-crosslinking and/or gelation (e.g. when exposed to heat). In addition, the second polymeric material should be a material that is capable of forming a hydrogel network with the first polymeric material.

In other words, the first polymeric material may be a material that can only form a gel with suitable mechanical strength when it is in the presence of a cross-linker (e.g. Ca²⁺) and the second polymeric material may be a material that can form a gel upon heating and cooling, albeit a mechanically weak one, that can interact with the first polymeric material in the cross-linked or uncross-linked state. The interaction of the two materials is what holds shape for the gel until the crosslinking agent (e.g. CaCO₃) is activated in the acidic gastric fluid (simulated or actual) and cross-links the first polymeric material.

The second polymeric material may be any suitable polymeric material with the properties discussed above. The second polymeric material may be a material that can undergo a sol-gel transition in the presence of a solvent (e.g. water) upon heating and subsequent cooling of the solution. The second polymeric material should also be able to interact with the first polymeric material to form a hydrogel network following this heating and cooling cycle. As such, the second polymeric material may be a hard-gel. That is a material that can retain a suitable degree of mechanical strength post thermal gelation. In embodiments that may be mentioned herein, the second polymeric material may be resistant starch, kappa-carrageenan, agarose, iota-carrageenan, cellulose, methylcellulose, ethyl cellulose, gelatine or combinations thereof. For example, the second polymeric material may be kappa-carrageenan.

As discussed in more detail herein, the first and second polymeric materials (e.g. an alginate and kappa-carrageenan) are not cross-linked when initially mixed together during the preparation of the pharmaceutical flotation device, but they are able to interact with each other to form a hydrogel network (e.g. an interpenetrating network (IPN) hydrogel structure) upon mixing. The second polymeric material (e.g. kappa-carrageenan) may be able to undergo gelling once it has been heated up and subsequently cooled down (thermal induced sol-gel transition) and the polymers are able to form a hydrogel in solution (e.g. an IPN), which can be subsequently dried to form the pharmaceutical flotation device (e.g. in the form of a receptacle raft). This pharmaceutical flotation device can hold its shape following ingestion for sufficient time until the crosslinking agent can generate crosslinks in at least the first polymeric material in the low pH of the stomach. For example, if the crosslinking agent is CaCO₃, then the low pH will generate calcium ions that can crosslink the first polymeric material (e.g. alginate). The crosslinking agent (e.g. CaCO₃) serves to further cross-link the network, which may enhance the mechanical strength of the gel raft.

Carrageenan (e.g. kappa- and iota-carrageenan) is a thermally responsive material that is able to form a hard hydrogel when cooled after it has been thermally activated via a pre-heating step.

The pharmaceutical flotation device may be provided in any suitable form. For example, the pharmaceutical flotation device may be provided in a first portion shaped as a hollow vessel and one or more second portions shaped as a cap and/or a plug, such that a pharmaceutical product can be deposited within the hollow vessel and sealed inside by the cap and/or plug. This form is suitable to receive a pharmaceutical product in any form—from raw active pharmaceutical ingredient through to a tableted formulation. Thus, in certain embodiments that may be mentioned herein, when the pharmaceutical flotation device is formed in the above manner, it may further comprise a pharmaceutical product.

Alternatively, the pharmaceutical flotation device may be formed to encapsulate a pharmaceutical product within its structure, so that it may adopt any suitable form. That is, a pharmaceutical product may be encapsulated within the polymeric matrix of the pharmaceutical flotation device.

When used herein, the term “pharmaceutical product” may refer to a pharmaceutical product in any form—e.g. from raw active pharmaceutical ingredient through to a tableted formulation. Examples of pharmaceutical products that may be mentioned herein include, but are not limited to, particles of one or more active pharmaceutical ingredients; particles of one or more active pharmaceutical ingredients and pharmaceutically acceptable excipients; a tableted formulation; a capsule formulation; drug-loaded microparticles; and drug-loaded fibre meshes.

While not necessary for the pharmaceutical flotation device to function, it may in certain embodiments further comprise an oil. Any suitable oil may be used herein. Examples of suitable oils include, but are not limited to olive oil, fish oil, corn oil, sunflower seed oil, grape seed oil, canola oil, avocado oil, coconut oil and combinations thereof. In certain embodiments that may be mentioned herein, the oil may be coconut oil.

In certain embodiments mentioned herein, the crosslinking agent may be poorly soluble in water. For example, the crosslinking agent may have a water solubility at 25° C. of less than 0.1 g/L, such as less than 0.02 g/L, such as about 0.013 g/L. Without wishing to be bound by theory, an advantage associated with the use of a poorly-soluble crosslinking agent is that it may help to avoid processing issues during the manufacture of the pharmaceutical flotation device. For example, if the crosslinking agent caused crosslinking during manufacture, then the resulting hydrogel may be more difficult to handle because the viscosity of the solution may significantly increase.

The crosslinking agent used herein may be any suitable crosslinking agent that can be activated when exposed to acidic conditions (e.g. pH 1 to pH 5, such as stomach acid conditions). In particular embodiments of the invention that may be mentioned herein, the crosslinking agent may be a bivalent metal carbonate. For example, the bivalent metal carbonate may be calcium carbonate. Without wishing to be bound by theory, use of the bivalent metal carbonate may have two functions: the first is the reaction of the bivalent metal carbonate in acidic conditions to release M²⁺ ions for the further crosslinking of at least the first polymeric material; and the second is to release carbon dioxide gas to produce effervescence that may help to boost floatation in the initial stages of administration.

The amount of the first and second polymeric materials may be any suitable amount relative to each other. For example, the wt:wt ratio of the first polymeric material to the second polymeric material may be from 1:10 to 10:1, such as about 1:1.

The amount of the crosslinking agent to the first polymeric material may be any suitable amount relative to each other. For example, the wt:wt ratio of the crosslinking agent to the first polymeric material may be from 1:10 to 10:1, such as about 1:1.

In particular embodiments that may be mentioned herein, the pharmaceutical flotation device may be one in which the polymeric matrix comprises:
sodium alginate;
kappa-carageenan; and
CaCO₃, where
   the wt:wt ratio of the sodium alginate:kappa-carageenan:CaCO₃ is about 1:1:1, optionally wherein the polymeric matrix further comprises coconut oil.

In embodiments herein, the hydrogel network may be an interpenetrating network.

In a further aspect of the invention, there is provided a method of forming a pharmaceutical flotation device as described hereinbefore, the method comprising the steps of:
(a) providing a solution mixture comprising:
   a first polymeric material;
   a second polymeric material;
   a crosslinking agent; and
   water, where the first polymeric material and the second polymeric material interact to form a hydrogel network;
(b) placing the solution mixture into a mould, solidifying the solution mixture and removing the water to provide a pharmaceutical flotation device.

The physical properties and the constituent components of the pharmaceutical flotation device made by this method are discussed hereinbefore and a discussion of these points will be omitted for brevity.

As noted hereinbefore, the second polymeric material may be one that undergoes a degree of gelation upon heating and cooling. Thus, in embodiments of the method, the solution mixture of step (a) may be heated to a temperature of greater than 40° C. for a first period of time and may then be cooled to about 40° C. for a second period of time before step (b) of the method is conducted. The mould in step (b) may be cooled to a temperature of less than 25° C. in order to assist in the cooling of the solution mixture post-heating.

As noted above, the amount of the first and second polymeric materials may be any suitable amount relative to each other. For example, the wt:wt ratio of the first polymeric material to the second polymeric material may be from 1:10 to 10:1, such as about 1:1. In the solution of step (a), the first and second polymeric materials may be present in any suitable quantity. For example, the maximum concentration of each of the first polymeric material and the second polymeric material in the solution mixture may be from 1 to 2 wt %/volume, such as about 1.5 wt %/volume. For the avoidance of doubt, the maximum concentrations listed above refer to the concentration of each of the components separately. As such, the maximum concentration of the first polymeric material may be 1 wt %/volume and the second polymeric material may have a maximum concentration of 2 wt %/volume.

As noted above, the amount of the crosslinking agent to the first polymeric material may be any suitable amount relative to each other. For example, the wt:wt ratio of the crosslinking agent to the first polymeric material may be from 1:10 to 10:1, such as about 1:1. In the solution of step (a), the crosslinking agent may be present in any suitable quantity. For example, the maximum concentration of crosslinking agent in the solution mixture may be from 1 to 2 wt %/volume, such as about 1.5 wt %/volume. As noted above, the maximum concentrations listed above refer to the concentration of each of the components separately.

The above parameters may be modified to provide a desired pharmaceutical flotation device. Some parameters that may be changed include, but are not limited to the following.

1. The type of hydrogel: the first and second polymeric materials should be able to withstand the pH switches in the stomach without undergoing any change in shape and structure. The mechanical strength, porosity density, pore volume and degradation rate of the raft can be adjusted by the choice of the first and second polymeric materials, as well as by the relative amounts of these materials in the eventual pharmaceutical flotation device.

2. Ratio of the hydrogels: the ratio of the first and second polymeric materials may also affect gel strength, floatation and the drug release profile for each active pharmaceutical ingredient placed within the pharmaceutical flotation device.

3. Concentration of hydrogel in the solution of step (a): the concentration of the first and second polymeric materials can be manipulated to control the strength of the raft and the release rates of any active pharmaceutical ingredient placed therein. Adjusting the concentration of each of the first and second polymeric materials in the solution, may allow for the modification of mechanical strength, porosity, and degradation rate of the pharmaceutical flotation device. For example, a higher concentration of the hydrogel blend (obtained by the combined concentrations of the first and second polymeric materials) may increase the mechanical strength of the pharmaceutical flotation device.

4. Concentration of the cross-linking agent: the cross-linking agent (e.g. calcium carbonate, potassium carbonate) aids in the further crosslinking of the first (and possibly the second) polymeric material only after it reaches the stomach, thereby increasing the hydrogel's mechanical strength and this may affect the release rate of each active pharmaceutical ingredient placed therein. If the crosslinking agent is a bivalent metal carbonate, then it may also generate CO₂, which may aid in the pharmaceutical flotation device floating in the stomach.

5. The type of floatation agent: the (optional) addition of oil may aid in the flotation of the pharmaceutical flotation device.

In a further aspect of the invention, there is provided a pharmaceutical formulation comprising a pharmaceutical flotation device as described herein and a pharmaceutical product. The active pharmaceutical ingredient in the pharmaceutical formulation may be any suitable active pharmaceutical ingredient that can be administered orally and is not particularly limited. As will be appreciated, two or more active pharmaceutical ingredients may be delivered by the pharmaceutical formulation (and pharmaceutical flotation device) disclosed herein.

As a proof-of-concept, the described pharmaceutical flotation device has been developed to release two different drugs, one hydrophilic and one hydrophobic, to demonstrate the versatility of the system. Any chronic disease condition, that requires multiple dosing per day and to be taken over a prolonged period would benefit from such a sustained-releasing drug delivery system to reduce the dosing frequency. Some examples include, but are not limited to, Type II diabetes, hypertension, hyperlipidaemia, Parkinson's disease, Alzheimer's disease, and other chronic conditions (i.e., mental disorders, stroke, HIV, tuberculosis, and lupus) that require regular and frequent medication.

The pharmaceutical flotation device described herein has the following advantages.

1. Ability to hold tablets, free drugs, or other pharmaceutical delivery systems.

2. Excellent floatability, of up to a month in simulated gastric fluid. In addition, multiple floatation mechanisms/aids may be combined (e.g. a porous nature, oil, a crosslinking agent which ensures the structure of the gel is preserved; the crosslinking agent may also double as a gas generating agent, which may help in the flotation of the device.

3. Controlled and sustained release of encapsulated drugs up to a week.

4. Produced through a one-step process that is readily scalable, enabling a high throughput due to the ease of fabrication. This may make use of a combination of materials that allows for interactions between the materials during the fabrication process, to strengthen and provide buoyancy to the eventual device.

5. Has enhanced drug loading efficiencies.

6. Use of food grade materials, which are considered to be non-toxic (i.e. the materials are generally recognised as safe (GRAS) by the US Food & Drug Administration).

7. Triggered degradation if required.

8. Can be adopted easily for any formulation. Translation from bench to bedside will be easier for any disease condition.

Further aspects and embodiments of the invention will now be discussed by reference to the following non-limiting embodiments.

### EXAMPLES

### Materials

Alg, KC, calcium carbonate (CaCO₃), pepsin, Ris, MT, sodium azide, lecithin, sodium chloride, sodium taurocholate hydrochloric acid (HCl), trisodium citrate, acetonitrile, phosphate-buffered saline (PBS), trifluoroacetic acid (TFA), and polycaprolactone (PCL, 80 kDa) were purchased from Sigma-Aldrich, Singapore. Polylactic acid (PLLA) was purchased from Purac. Coconut oil and olive oil were store-bought food grade products.

### Analytical Techniques

### Electron Microscopy

For characterization using electron microscopy, the samples were prepared by snap-freezing the dry hydrogel raft in liquid nitrogen and cutting with a scalpel to obtain surface and vertical cross sections; the liquid nitrogen helps harden the hydrogel raft, which allows for easier cutting. The obtained sliced samples were freeze-dried overnight before imaging with Thermionic SEM JEOL JSM-6360.

### Scanning Electron Microscopy (SEM)

For SEM (Thermionic SEM JEOL JSM-6360) imaging, the samples were attached to the SEM stub via carbon tape and secured with copper tape around all sides of the sample. Thereafter, the samples were coated with platinum for 45 s at 20 keV before installation in the SEM chamber.

### High-Performance Liquid Chromatography (HPLC)

HPLC was carried out on an Agilent 1100 series HPLC. Ascentis® C18 column was used.

### Example 1. Fabrication of the Raft

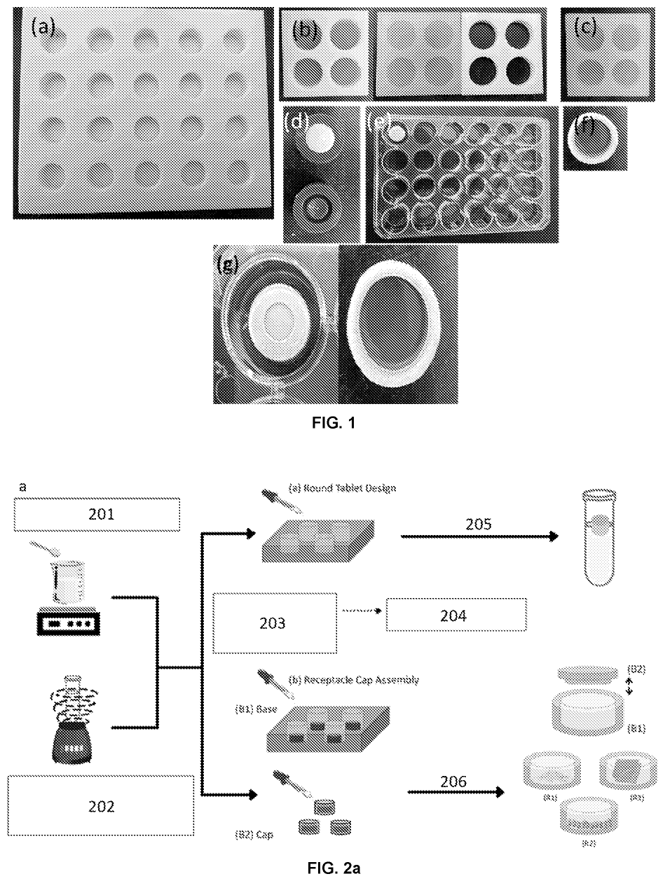

The floating delivery system was prepared using a combination of two biopolymeric hydrogel materials. Biopolymeric hydrogels that were approved as GRAS materials were used for this fabrication. All other materials used in fabricating the raft, such as cross-linking agents and flotation agents are also GRAS materials. The raft has been fabricated using a single step process that is easily scalable. Biopolymeric hydrogels that are resistant to swelling and degradation in the gastric pH have been used. A stock solution of the hydrogel premix was prepared by mixing the hydrogel materials and the cross-linking agent. Once the premix was stirred homogenously, it was heated in a water bath while being stirred to enable the hydrogel to dissolve completely. Following this, premix was allowed to cool under stirring. Once the solution cooled down, it was diluted to the desired concentration by the addition of a flotation agent (i.e. oil) and water. The addition of the flotation agent and water at this stage also allows for the incorporation of hydrophobic or hydrophilic drugs to the premix respectively if they were to be embedded in the raft. The final solution was then vortexed at max speed for 15 s, followed by sonication in a bath sonicator for 1 min. This vortex and sonication cycle was repeated for five times. The hydrogel solutions were then poured into the respective pre-chilled moulds (FIG. 1) and frozen for two hours before freeze drying for 24 h. Once dried, the raft was obtained by de-moulding and can be used to hold the various drugs, tablets, MPs or FMs.

Hydrogels are made of hydrophilic polymer networks that can swell and retain large quantities of water without undergoing dissolution. They are used in diverse applications in day-to-day life from contact lenses to hygiene products. They also find extensive use in the biomedical sector, mainly in drug delivery and tissue engineering. The GRDDS described here uses two such hydrogels, Alg and KC, both natural polymers, classified to be generally recognized as safe (GRAS). The choice of material is important, not only for the functionality of the drug delivery device, but also to alleviate safety concerns as the delivery system would be subject to prolonged use for extended release. Among the variety of matrix materials, Alg, a negatively charged linear polysaccharide consisting of 1→4 linked β-(D)-guluronic and α-(L)-mannuronic acids derived from brown algae and KC, a linear hydrophilic sulphated galactan extracted from marine red algae, were particularly preferred. Alg was chosen for its biocompatibility, low cytotoxicity and ionic gelation via cationic crosslinking. KC was used as a complementary biopolymer to enhance the mechanical strength and reduce the pore sizes of the matrix. Like Alg, KC is frequently used for encapsulation due to its biocompatibility and low cytotoxicity. The similar gelling mechanism between KC and Alg enhances their synergism and provides for better mechanical properties. In addition, KC is able to undergo sol-gel transition under heat treatment which further increases the mechanical strength of KC-Alg hydrogel. Both Alg and KC are used widely in the food industry as thickening and stabilizing agents. It is hypothesized that combining the two materials allows them to interact via cross-chain entanglement to form hydrogel networks with high porosity and enhanced mechanical strength. A crosslinking agent such as calcium carbonate and a low-density flotation aid have been included in the fabrication. This further improves the functionality of the delivery system, resulting in an ultra-long floatable hydrogel raft.

In addition to the choice of material, which was a key determinant to achieve long floatability under gastric conditions, three other flotation mechanisms were incorporated to ensure the buoyancy of the delivery system.

### Manipulation of Process Parameters

1. The type of hydrogel: a biopolymeric hydrogel (single or in combination) that can withstand the pH switches in the stomach without undergoing any change in shape and structure. Further modification can be made by adding any of the other listed hydrogels (e.g. Pectin, alginate, resistant starch, k-carrageenan, agarose, iota carrageenan, cellulose, methylcellulose, ethyl cellulose, and gelatine) to adjust mechanical strength, porosity density, pore volume and degradation rate of the raft.
2. Ratio of the hydrogels: When using combination materials, the ratio of combining the two materials can be manipulated to control gel strength, flotation and the drug release profiles.
3. Concentration of hydrogel: it can be manipulated to control the strength of the raft and the drug release rates. Adjusting the concentration of each of the hydrogel solution allows modification of mechanical strength, porosity, degradation rate of the raft. A higher concentration of hydrogel blend increases mechanical strength of hydrogel raft.
4. Concentration of the cross-linking agent: the cross-linking agent (e.g. calcium carbonate and potassium carbonate) aids in cross linking the alginate only after it reaches the stomach, thereby increasing the gel strength. It also generates CO₂ that aids in flotation. It can be manipulated to control the strength of the raft and the drug release rates.
5. The type of flotation agent: the addition of oil aids in the flotation of the raft. Any type of oil (e.g. olive oil, fish oil, corn oil, sunflower seed oil, grape seed oil, canola oil, avocado oil, and coconut oil) can be used for this purpose.

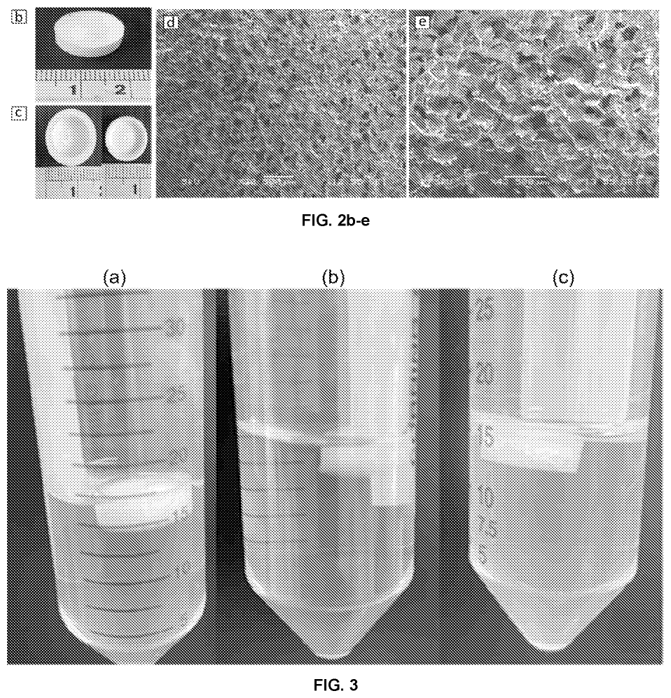

The fabrication of the delivery system was achieved using a simple mould casting technique. The fabrication process uses pre-set moulds into which the hydrogel mix was poured to obtain the hydrogel rafts as shown in FIG. 2. As shown in FIG. 2, the schematic showing the fabrication process 200 of the raft includes the steps:
201. heat KC-Alg solution to 80° C. for 5 min and stir at 450 rpm;
202. dilute KC-Alg (cooled to 40° C.) solution with coconut oil (preheated at 37° C.) and water. Sonicate and vortex solution homogeneously;
203. pipette final hydrogel solution into pre-chilled moulds (−20° C. for 30 min);
204. refrigerate at −20° C. for 2 h and freeze-dry overnight;
205. for characterization:
   (1) flotation;
   (2) SEM;
   (3) simulated gastric pH switches; and
   (4) triggered degradation; and
206. for drug release studies on Ris & MT:
   (R1) free drug (FD, powdered);
   (R2) microcapsules; and
   (R3) FM.

Herein, the described method not only produces a dry hydrogel raft capable of prolonged flotation within the gastric environment, but also uses a mild and scalable fabrication technique that can be applied to a variety of drugs. The materials used for the raft were chosen after careful consideration to fulfill the objectives of the desired GRDDS. Unlike other methods of producing floatable delivery systems, high temperature and compression forces are not required in this technique. The advantage of using low fabrication temperature is that it allows the loading of thermally sensitive drugs that could degrade under high temperature. Also, the use of compression forces during fabrication could be energy-intensive and costly. On the other hand, only simple and economical apparatus are required for this hydrogel raft that can be easily scaled up for high throughput fabrication.

### Example 2. Preparation and Flotation Study of Different Hydrogel Combinations

To demonstrate the role of KC, Alg, oil and CaCO₃ in the flotation of the raft, samples such as rafts made of KC, Alg or KC-Alg mix without coconut oil and CaCO₃ (control), with either coconut oil or CaCO₃ and containing both coconut oil and CaCO₃ (mix) (as shown in Table 1) were prepared by making slight variations to the protocol described in Example 1.

### Flotation Studies

Tablet type rafts were used for the flotation studies. The flotation of the raft was tested under simulated gastric conditions. The samples were then added to FaSGF prepared by adjusting the pH of water to 1.2 using 37% HCl. All flotation studies were conducted in a shaking incubator (150 rpm) at 37° C. in triplicates. The samples were allowed to float until they showed visible degradation by disintegrating into pieces, since none of the samples sank.

TABLE 1
Flotation studies for KC-Alg raft in SGF.

| Hydrogel | Flotation | Float | | |
|---|---|---|---|---|
| Combination | Lag | Duration | | Remarks |
| KC (control) | — | <1 | h | Gel dissolved completely |
| Alg (control) | — | 1.5 | h | Sunk after initial 5 min, |
| | | | | floated again and sustained |
| | | | | floating for ~1 h before |
| | | | | sinking again; gel shrinkage |
| KC-Alg | — | 25 | days | Gel remained intact with |
| (control) | | | | initial shape |
| KC (oil) | — | <1 | h | Gel dissolved completely |
| | | | | (residual oil turns |
| | | | | SGF cloudy) |
| Alg (oil) | — | 25 | days | Gel shrinkage |
| KC-Alg (oil) | — | ~19-21 | days | Gel remained intact with |
| | | | | initial shape |
| KC (mix) | — | <1 | h | Gel dissolved completely |
| Alg (mix) | — | 24 | days | Gel shrinkage |
| KC-Alg | — | 24 | days | Gel remained intact with |
| (mix) | | | | initial shape |
| KC (CaCO₃) | — | <1 | h | Gel dissolved completely |
| Alg (CaCO₃) | — | 31 | days | Gel shrinkage |
| KC-Alg | — | 31 | days | Gel remained intact with |
| (CaCO₃) | | | | initial shape |

### pH Switching and Dissolution Studies

### PH Switching

The raft was subject to four pH switches in a day using FaSGF and FeSGF at pH 5. FaSGF was prepared by dissolving 80 μM sodium taurocholate, 20 μM lecithin, 0.1 mg/ml pepsin and 34.2 mM sodium chloride in distilled water with 0.02% (W/V) sodium azide as a preservative. PBS at pH 5 was used as the FeSGF. pH switching was carried out by switching the raft between FaSGF (fasted-state SGF that is enzyme loaded) and FeSGF (fed-state SGF which is PBS adjusted to pH 5.0) every 1.5 h interval.

Fresh medium were used for each switch and a total period of 4 FaSSGF and 3 FeSSGF periods were conducted each day.

The sequence was Fa-Fe-Fa-Fe-Fa-Fe-Fa.

### Dissolution

The degradation of the raft in the presence of a chemical trigger was proven using trisodium citrate as the dissolution agent. Four different concentrations of sodium citrate (0.025 M, 0.05 M, 0.1 M, and 0.2 M) were prepared by dissolving the corresponding amount of trisodium citrate in distilled water. The raft that was exposed to SGF for 24 h was then added to each of these solutions and incubated at 37° C. and shaken at 150 rpm for 5 min.

### Estimation of Porosity and Pore Size

The solvent resaturation method was used for the estimation of porosity of the raft (Unosson, J. E., Persson, C. & Engqvist, H., J. Biomed. Mater. Res. Part B 2015, 103B, 62-71). Briefly, the dimensions and weight (W_{d}) of the tablet raft were measured in dry form. The rafts were then submerged in a known volume of ethanol overnight. The rafts were then weighed to measure the weight after saturation of the pores with ethanol (Wₛₐₜ). The porosity of the raft was obtained based on the equation below. The average pore size was calculated using ImageJ software from the SEM micrograph of the cross-section of the rafts

$$ϕ ( \% ) = ( \frac{( W s a t - Wd ) / Density of the solvent}{Apparent volume of the raft} ) * 100$$

### Results and Discussion

The freeze-dried hydrogel rafts were examined using electron microscopy to understand the surface and cross-sectional morphology. Both the surface and internal morphologies of the raft showed interconnected porous structures, as observed from the scanning electron micrographs in FIGS. 2d and e. While the surface of the raft was observed to contain fewer pores compared to the interior, this was later attributed to the clogging of some of the surface pores due to the addition of oil during the fabrication process. Samples fabricated without oil were uniformly porous than those with oil. The floatability of the raft, an essential characteristic to promote gastro-retention, was tested by placing them in FaSGF. The results of the flotation study have been tabulated in Table 1. Four samples, KC-Alg (control), KC-Alg (oil), KC-Alg (mix) and KC-Alg (CaCO₃), showed good floatability over a prolonged period without undergoing any change in the initial shape. The floatability of the raft is attributed to three different mechanisms of flotation that have been incorporated into the fabrication design of the raft. The porous nature of the raft, the addition of a low-density flotation aid such as oil, and the presence of a gas-generating agent, which also served to cross-link the alginate under acidic pH conditions, are the three mechanisms that imparted buoyancy to the raft.

Whereas all four sample groups previously mentioned contain one or more of the three flotation mechanisms, the system that incorporated all three flotation mechanisms was also found to exhibit the utmost resistance against disintegration when mimicking in vivo conditions (i.e. alternating pH switch to mimic the fasted and fed states in the stomach). Dual gel network between Alg and KC produces a network with high porosity that lowers overall density of the delivery system, hence enabling flotation. The second flotation mechanism is the use of an oil. The dispersion of a low-density liquid such as coconut oil throughout the hydrogel raft system further reduces the initial density of the raft and boosts flotation. As the oil diffuses out with time, micropores are created, which further retains a lowered density for a longer time, not only enhancing flotation but also increasing the rate of water uptake for drug diffusion. The third flotation aid is the addition of an effervescent component such as CaCO₃ which reacts in the low pH of gastric environment, serving to cross-link the hydrogel and enhance the mechanical properties of the raft in the gastric environment and to generate CO₂ as effervescence to further aid in flotation of hydrogel raft in the stomach.

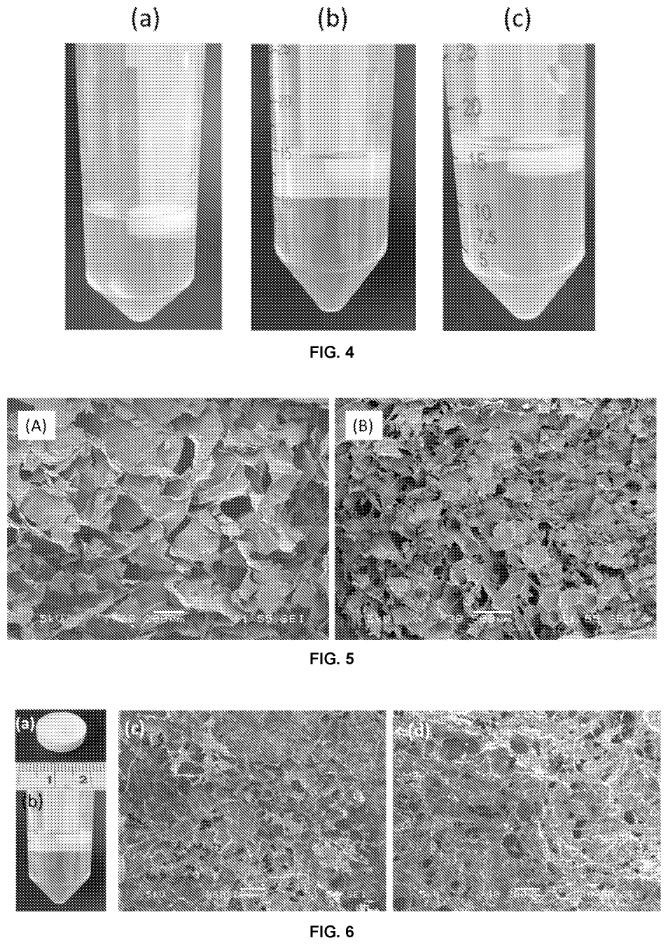

In the experiment where the raft was subject to four pH switches in a day using FaSGF at pH 1.2 and FeSGF at pH 5, KC-Alg (control) and KC-Alg (oil) disintegrated when they were switched from FaSGF to FeSGF due to the dissolution of uncrosslinked alginate at pH 5. Both KC-Alg (mix) and KC-Alg (CaCO₃) withstood the pH switches for seven days as shown in FIGS. 3-4. Nonetheless, KC-Alg (mix) was chosen for further studies because of an additional flotation mechanism (oil) to ensure the floatability of the raft. In addition, the presence of oil in the pores as observed from SEM, aids in mitigating burst release of hydrophilic drugs while facilitating release of hydrophobic drugs. The pore size of the KC-Alg (mix) raft was calculated to be 391.019±0.003 μm and the porosity measured using the ethanol resaturation method was 28.23±8.01% (FIG. 5).

### Example 3. Preparation and Characterization of KC and Alg Based Raft (KC-Alg (Mix))

Different moulds used for the fabrication process have been shown in FIG. 1. Alg and KC were used as the hydrogel materials. A stock solution of the hydrogel premix was prepared by mixing 1.25% (W/V) Alg, 1.25% (W/V) KC and 1.25% (W/V) of the cross-linking agent CaCO₃ in water. The hydrogel premix was allowed to stir overnight at 450 rpm. Once the premix was stirred homogenously, it was heated in a water bath to 80° C. for 5 min, while being stirred at 450 rpm to enable the KC to dissolve completely. Following this, the premix was allowed to cool to 40° C. under stirring. Once the solution cooled down, the concentration of the premix was adjusted to 1% (w/v) by the addition of coconut oil (10% v/v) preheated to 37° C. and water.

The final solution was then vortexed at maximum speed for 15 s, followed by sonication in a bath sonicator for 1 min. This vortex and sonication cycle was repeated five times. 1 ml of the final hydrogel solution was pipetted into a specially moulded size 24′ well-plate (pre-chilled on ice for at least 30 min) (see FIG. 1). The samples were frozen immediately at −20° C. for 2 h and then freeze-dried overnight. Freeze drying allows for the removal of water from the hydrogels creating porous structures that provide buoyancy. Finally, the dried samples were demoulded and stored under dry conditions before being used for further tests in the following examples.

### Example 4. Flotation of KC and Alg Based Raft

The pH switching and flotation of the raft (prepared in Example 3) was tested by following the flotation studies and pH switching protocols in Example 2.

### Results and Discussion

The raft floated instantaneously and continued to float for 24 days without any changes in the initial shape as shown in FIG. 6b. The raft was subject to four pH switches in a day using FaSGF and FeSGF at pH 5 as shown in FIG. 4. The raft withstood the pH switch for 7 days, after which the experiment concluded. It is important to note that the hydrogel rafts were still capable of flotation and pH switching.

### Example 5. Compression and Buoyancy Tests

The ability of the raft (prepared in Example 3) to withstand the peristaltic forces and the pressure due to the presence of food was tested using a compression test. The raft was subject to four cycles of pH switching each day (by following the pH switching protocol in Example 2) and subject to a compression test at the end of the switches, after which they were tested for buoyancy.

### Compression Test

MTS Criterion® Model 43 Electromechanical Universal Test System was used. The gel raft was placed under the compression plates and the test was carried out by using a 50 N load at a speed of 10 mm/min under 50% strain, and the compression study results were obtained. After the gels were compressed, they were resubmerged into SGF and were observed to regain to pre-compression shape. The gels were observed for any breakage and post-compression floatability in FaSGF.

### Buoyancy Test

The raft was added to the SGF solution to observe the floating lag time, total floatation duration and signs of degradation. Upon addition of raft to the 50 ml centrifuge tube containing SGF (pH 1.2), the tube was placed in a shaking incubator at 37° C. and 150 rpm.

### Results and Discussion

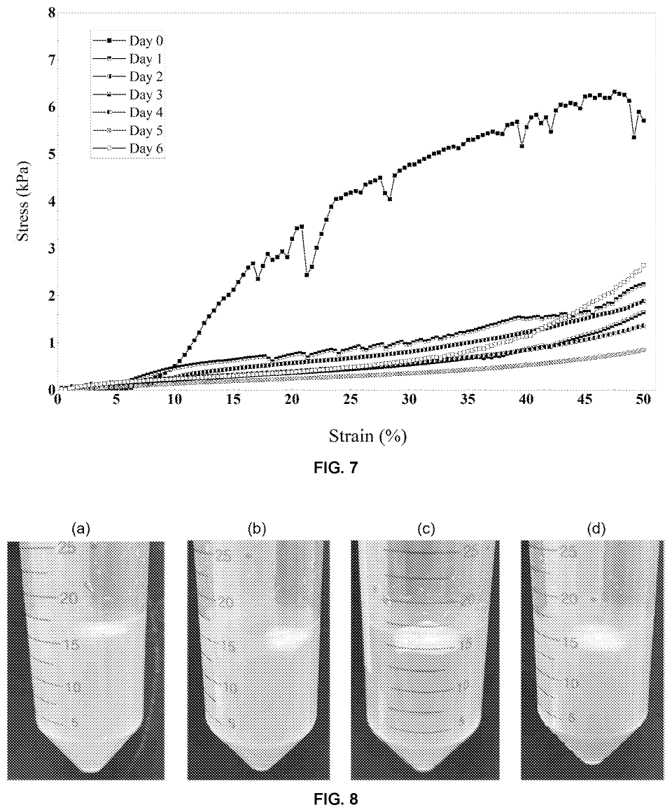

The compression test results are shown in FIG. 7. All samples were able to withstand the load and could float back well after the compression test.

The KC-Alg combination hydrogel enhanced the integrity of the delivery system as KC and Alg are able to interact via cross-chain entanglement to form a double hydrogel network with high porosity and enhanced mechanical strength. The resultant hydrogel system is able to retain the high anti-swelling property of Alg while possessing higher mechanical strength from the crossed network. This improved mechanical property of a combination material helps to retain the structure of the hydrogel delivery system for a prolonged duration even with changes in gastric pH (from fasted to fed and vice versa).

### Example 6. Triggered Dissolution of the KC-Alg Raft

Since the raft (prepared in Example 3) can float in SGF for two weeks, as ascertained from the pH switch experiments in Example 4, the degradation of the raft on a trigger is desired, with the aim of dissolving the raft once drug release is completed or in the case of an emergency.

### Results and Discussion

The rafts dissolved entirely when trisodium citrate was added. A range of concentration of the trisodium citrate solution was tested to ascertain that solubility of the hydrogel is attained at a safe concentration of the trigger. An indicative photo is shown in FIG. 8. Trisodium citrate is a GRAS material that does not pose any toxicity to the human body with a known toxicity of LD50 oral>5000 mg/kg. It can dissolve away cross-linked alginate which eventually leads to the dissolution of KC, all of which can then be safely passed out of the body.

The ability of the raft to dissolve upon trigger is an added advantage to ensure the raft can be removed completely from the body once the drug release is complete. This incorporates a safety feature which is not observed for some long releasing delivery systems.

### Example 7. Fabrication of MPs

The MPs used for the encapsulation of MT and Ris were fabricated using a double-emulsion method (WO2019027372A1). Briefly, the primary water phase was prepared by dissolving 50 mg of casein and 2 mg of sodium chloride in 1 mL of distilled water. The polymer solution (oil phase) was prepared by dissolving 0.3 g of PLLA and 0.1 g PCL in 5 mL of dichloromethane (DCM). For the loading of drugs, the hydrophilic drug MT was added to the casein solution, while for the encapsulation of Ris, it was added to the polymer solution. The primary emulsion was prepared by introducing the casein/drug solution drop-wise into the polymer solution with 10 μL of olive oil under magnetic stirring. This emulsion was then further dispersed into a 0.25% (w/v) of polyvinyl alcohol (PVA) solution (pH 2.5, 50 mL) containing DCM (1 mL) to form a water/oil/water (W/O/W) emulsion, with an over-head stirrer (Calframo BDC1850-220). The stirrer was operated at 300 rpm for 10 min to evaporate the DCM. The resultant emulsion was quickly poured into a round bottom flask filled with 0.25% (w/v) PVA solution (150 mL) and transferred to a rotary evaporator to solidify the microcapsules. The microcapsules obtained were washed with distilled water, before freeze drying for further use.

### Characterisation

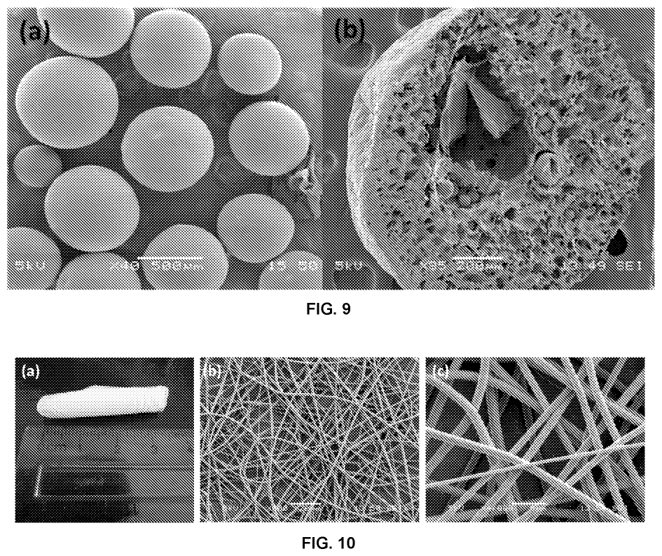

FIG. 9 shows the surface and cross-sectional morphology of the MPs.

### Example 8. Fabrication of FM

The FM used for the encapsulation of MT and Ris were fabricated using the electrospinning method. A polymer solution containing 375 mg of PLLA and 125 mg of PCL (80 kDa) was prepared by dissolving the polymers in DCM. The solution was then filled into a plastic syringe and spun into fibers using the NANON electrospinning setup, at a voltage of 23 kV and a flow rate of 1.5 mL/hr using a 25G blunt end needle. The fibers on collected on aluminium foil and peeled off once the solvent evaporated, to create the FM as shown in FIG. 10.

Conventional floating drug delivery systems involve systems whereby only free drugs can be encapsulated, and most often lack controlled release capabilities. The proposed hydrogel raft can not only encapsulate free drugs, but also be used to contain other drug carriers, such as MPs and FM, as drug-encapsulation matrixes. In order to demonstrate the ability of the raft to control the release of drugs, two different model drugs, MT and Ris, were used in Examples 9 and 10. Both of the drugs were used in three different forms (FD, MP, and FM encapsulated with drugs).

### Example 9. Release of a Hydrophilic Drug from the Raft

MT (log P 1.76) is a water-soluble drug used in the treatment of hypertension, usually prescribed to be taken 1-3 times in a day. MT was used as the model hydrophilic drug to demonstrate the ability of the raft to suppress burst release of such drugs. Three different forms of the drug were used: 1. free drug; 2. drug encapsulated MPs (FIG. 9); and 3. drug encapsulated FMes (FIG. 10).

### MT-Encapsulated Rafts

Receptacle cap type rafts were used for the release studies as the receptacle allows the loading of the FD, MP, and FM. The receptacle cap assembly also ensures that the system can be adopted for other drugs easily. KC-Alg hydrogel raft (prepared in Example 3) were made into a receptacle cap type assembly using the mould shown in FIGS. 1d and f. This design was used to prevent the interaction of drugs with water in the hydrogel. 100 mg of free drug MT was weighed in triplicates and added to the receptacle of the raft before closing the lid to complete the assembly. Similarly, 20 mg of the MPs (prepared in Example 7) or one FM (prepared in Example 8) was weighed in replicates and added to the receptacle of the raft and closed with cap to obtain the raft containing MT in different forms. Drug forms that were not encased in the raft served as controls.

### Release Studies of MT

Required amounts of FD, MP, or FM were added to the receptacle of the raft, and the cap was used to close the structure to obtain FD encased in raft (FD-R), MP encased in raft (MP-R), and FM encased in raft (FM-R). This raft assembly was then tested under simulated gastric conditions using FaSGF in a shaking incubator (150 rpm) at 37° C. All experiments were carried out in triplicates. At specified time points, the release media were collected and replaced with an equal volume of fresh FaSGF. The collected samples were analyzed for the drugs using HPLC. For MT, acetonitrile:PBS (60:40) (PBS adjusted to pH 4.7) was used as the mobile phase at a flow rate of 0.8 ml/min and detected at 244 nm. Each drug form, without the raft, served as a control. To elucidate the mechanism of drug release, the release data were fitted to mathematical models.

### Results and Discussion

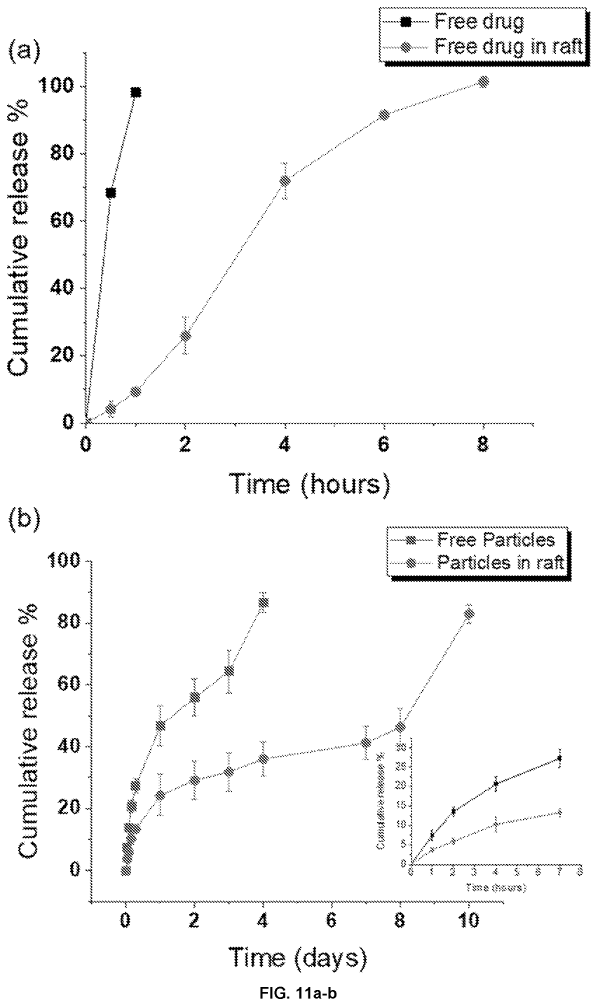

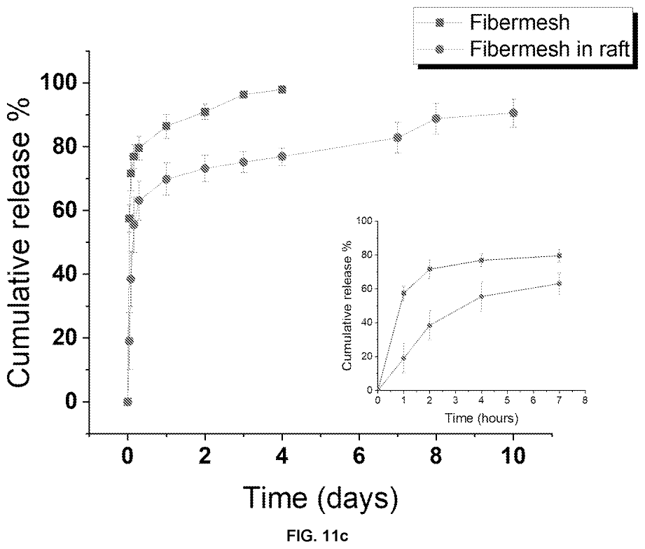

In vitro drug release studies ensued to ascertain the controlled release ability of the raft. All three forms of the drug showed a suppression in the initial burst release (in the first 2 h) when encased in the raft as seen from FIG. 11. The release of free drug MT was sustained over eight hours in the raft when compared to the uncased drug which was dissolved completely in one hour, indicating its unsuitability for multiday release. FM was used as a matrix to encapsulate the drug to prolong release. While FM required 4 days for complete release, FM encased in raft took 10 days to release all of the drugs. A huge initial burst release of 80% and 60% for MT in the first 24 h from FM and FM encased in raft, respectively, was observed. Up to 60% of the drugs were released in the first 24 h for FM encased in raft, which was only a 20% reduction compared to the free FM. The huge burst release is attributed to the porous nature of the fibers and also the narrow diameter allowing for ease of drug diffusion (Wu, J. et al., J. Control. Release 2020, 320, 337). To overcome the huge burst release, MT was encapsulated in MP, as the next matrix. MP provided a sustained release of the drug over four days but had poor floatability (only 24 h). By encasing MP within the raft (MP-R), the drug release was prolonged over a 10-day period, as seen from FIG. 11, while maintaining its floatability. Thus, in the case of MPs, the raft encased ones showed a sustained release of MT over 10 days. This offers promise that such a combination of microparticle matrix in the raft could be applied to hydrophilic drugs to circumvent any initial burst release.

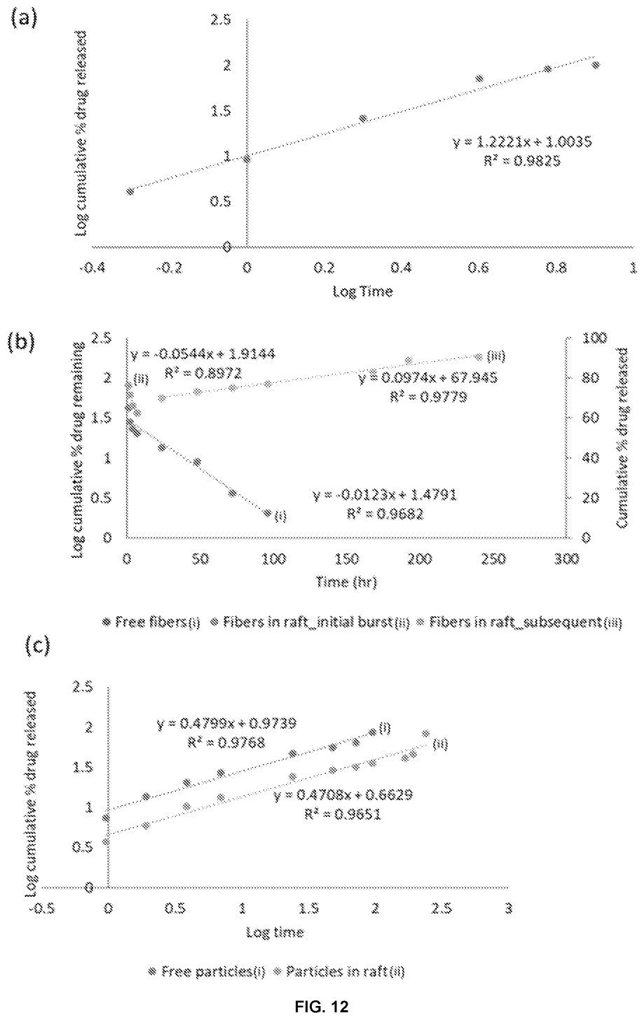

The release of FD-R fits well into the Korsmeyer-Peppas model with a correlation value (r²) of 0.9825 and n>1 (exponent of release), indicating a non-Fickian transport (FIG. 12a). The water solubility of the drug and the porous nature of the raft facilitated the diffusion of the drugs with the influx of the release medium into the raft. Drug release kinetics changes with the inclusion of a matrix (i.e. MP or FM). The drug release from FM fits into the first-order release kinetics with an r² value of 0.9682, where the release mechanism is mainly concentration-dependent diffusion. For FM-R, the release could be modelled in two stages, with the initial burst release (1-7 h) fitting into the diffusion-dependent first-order kinetics (r²=0.8972) and subsequent release (after 24 h) fitting well into zero-order kinetics (r²=0.9779) (FIG. 12b). The mathematical modelling of the release from MP fits well into the Higuchi model with a correlation of 0.9774, similar to the release kinetics of drugs from such floating polymer microparticles (Baek, J.-S. et al., Small 2016, 12, 3712). The Korsmeyer-Peppas model was used to understand the mechanism of release as it is a more comprehensive model, developed based on the Higuchi model, to study drug release from polymeric matrixes (M. L. Bruschi, “Mathematical Models of Drug Release,” in Strategies to Modify the Drug Release from Pharmaceutical Systems, ed. by M. L. Bruschi (Woodhead Publishing, Sawston, 2015), pp. 63-86). The release data from MP and MP-R showed good fit into the Korsmeyer-Peppas model with an r² value of 0.9768 and 0.9651, respectively. Based on the n value of 0.4799, obtained from the equation in FIG. 12c, for MP, the mechanism is being governed equally by both diffusion and the rearrangement of polymeric chains, leading to time-dependent anomalous transport (0.43<n<0.85 for spheres). For MP-R, the release is governed by a combination of diffusion and swelling.

### Example 10. Release of a Hydrophobic Drug from the Raft

Ris (log P 3.27) is an antipsychotic drug which is insoluble in water, is used in the treatment of a range of mental/mood health disorders such as schizophrenia, bipolar disorder and autism, and is usually prescribed over a long term. Ris was chosen as the model hydrophobic drug to demonstrate the ability of the raft to suppress burst release of such drugs. Four different forms of the drug were used: 1. free drug; 2. commercial tablet; 3. drug encapsulated MPs; and 4. drug encapsulated FMes. The release studies of Ris-encapsulated rafts were performed by following the release studies protocol in Example 9 except water:acetonitrile (70:30) with 0.1% TFA was used as the mobile phase at a flow rate of 1 ml/min and detected at 280 nm for Ris. Each drug form, without the raft, served as a control.

### Ris-Encapsulated Rafts

Receptacle cap type rafts were used for the release studies as the receptacle allows the loading of the FD, MP, and FM. The receptacle cap assembly also ensures that the system can be adopted for other drugs easily. KC-Alg hydrogel raft (prepared in Example 3) were made into a receptacle cap type assembly using the mould shown in FIGS. 1d and f. 4 mg of free drug Ris was weighed in triplicates and added to the receptacle of the raft before closing the lid to complete the assembly. One tablet each of Risperidone USP (4 mg) (American Remedies Healthcare Pte Ltd), 20 mg of the MPs (prepared in Example 7) or one FM (prepared in Example 8) was added to the receptacle of the raft and closed with cap to obtain the raft containing Ris in different forms. Drug forms that were not encased in the raft served as controls.

### Results and Discussion

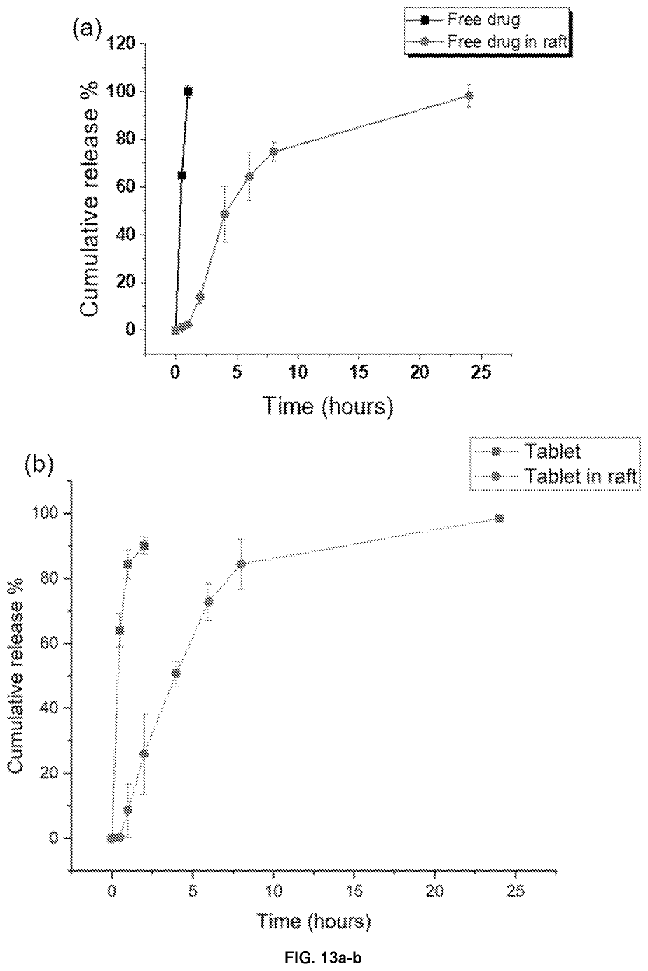

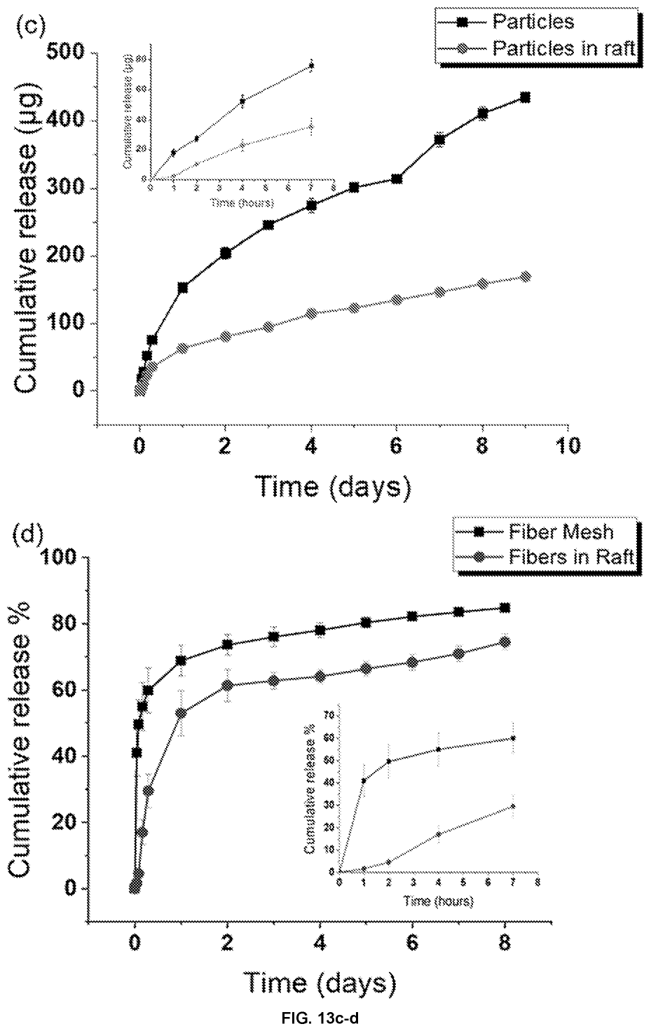

All four forms of the drug showed a suppression in the initial burst release when encased in the raft as seen from FIG. 13. The release of free drug and tablet form of Ris, was sustained over 24 h in the raft when compared to the uncased drug which was dissolved completely in 1 h. FD-R that releases the drug over 24 h could be used in place of such a formulation to provide a sustained release of the drug.

In the case of MPs and FM, while both the free and encased forms showed a sustained release of the drug, the encased forms were able to suppress the initial burst more effectively and most importantly, they were able to float in the release medium during the entire study period while the free forms sank after 24 h. Similar to the release of MT from FM, Ris-loaded FM also showed initial burst release with 50% of the drugs releasing in the first 24 h despite the raft. Ris has been shown to interact with hydrophobic polymers leading to hydrophobic interactions that retard release rates (Korzhikov, V. et al., J. Microencapsul. 2016, 33, 199; and Prieto, M. J. et al., PLoS ONE 2014, 9, e90393). This interaction was not much pronounced in the case of the FM due to the porous nature of the fibers.

The drug polymer interaction was quite conspicuous for MP where only 25% and 20% of the drug was released after 10 days from MP and MP-R, respectively. This indicates that the microparticles require a longer time to achieve complete release of Ris, concurring with previous studies that showed prolonged three months release of Ris from PLA microparticles implanted in rats (Yan, X., Wang, S. & Sun, K., Pharmaceutics 2021, 13, 1210). The present formulation could provide for such applications through the oral route by combining the drug-encapsulated microparticles and the hydrogel raft.

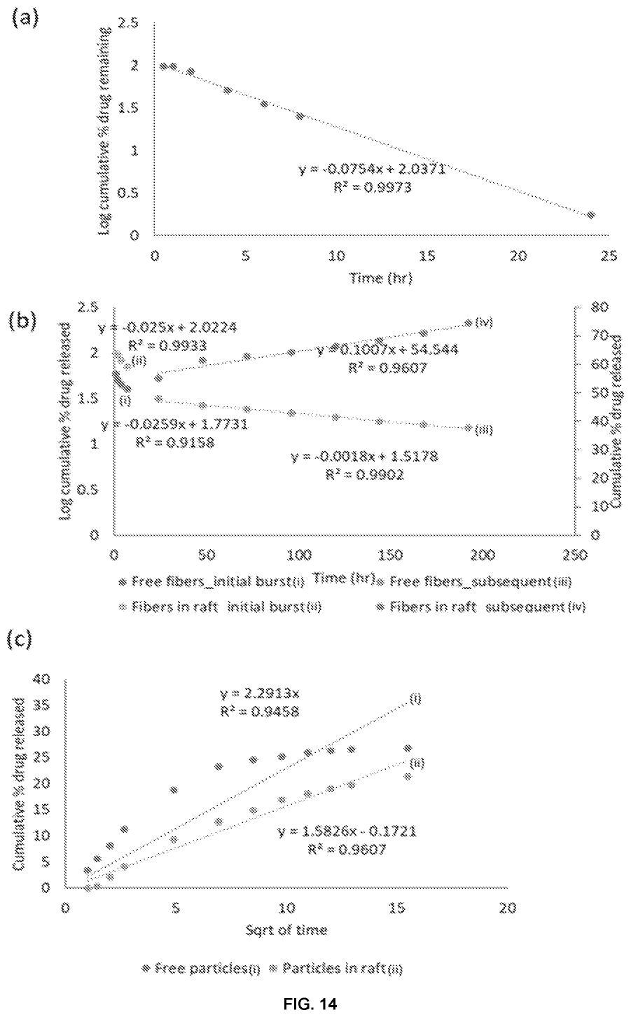

The mathematical modeling of the release kinetics of Ris release from the raft follows first-order release kinetics with an r² value of 0.9973, with concentration-dependent diffusion being the main mechanism of drug release (FIG. 14a). The release kinetics for both FM and FM-R can be fitted into two stages as seen from FIG. 14b. For FM, both the initial burst (1-7 h) and the subsequent release (from 24 h) follow first-order kinetics with a correlation of 0.9158 and 0.9902, respectively. The initial burst release from FM-R follows first-order kinetics with an r² value of 0.9933, and the subsequent release correlated with zero-order kinetics with an r² value of 0.9607. The mechanism of Ris drug release from MP is similar to that of MT, where drug release from MP fits well into the Higuchi model with a correlation of 0.9458, and the drug release from MP-R fits with a correlation of 0.9607 (FIG. 14c).

### Example 11. Release Rate Constant (k_{H}) for the Drug Release from Different Formulations

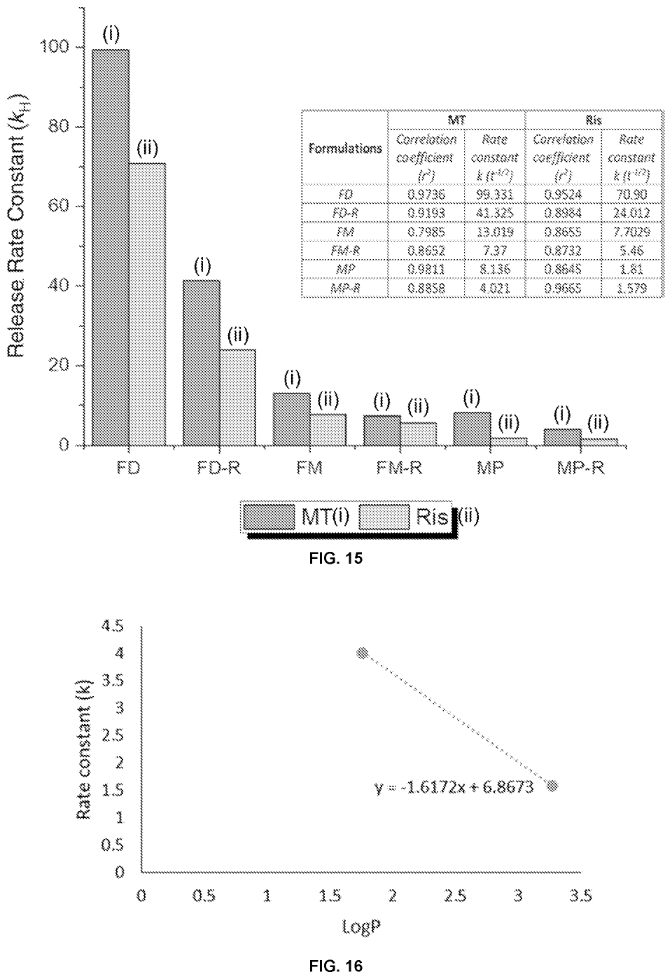

The k_{H} for the drug release from the different formulations was obtained using the Higuchi equation. The drug release profiles for both drugs in all formulations showed a reasonably good fit to the Higuchi equation. Hence, it was used to obtain the rate constant for the purpose of comparison. A low value for the rate constant indicates that the drug is being released in a sustained manner. The presence of the raft lowered the value of k_{H}, irrespective of the log P of the drugs as seen from FIG. 15. Encasing the free MT in the raft lowered k_{H} by twofold, whereas the presence of a matrix such as FM or MP decreased k_{H} by five and ten times, respectively. In addition, as the log P value increased, the rate constant was further decreased, suggesting that the raft suppressed the release of hydrophobic drugs more than that of the hydrophilic ones. This was clearly observed by the decrease in the release rate constant by 15 times for Ris-loaded MP-R when compared to FD-R. Based on this, it could be concluded that the encapsulation of the drugs in the MPs and encasing these in the raft could provide a sustained release of the drugs. It could also be projected that the higher the log P value of the drugs, the longer the drug release would be prolonged.

The above disclosure on the release of MT and Ris shows that the MP-R formulation provides the most optimal release profiles for the sustained release of drugs over multiple days. An empirical equation to predict k_{H} based on log P of drugs could be obtained by plotting the log P values of the two model drugs and the Higuchi rate constant (k_{H}) for MP-R (FIG. 16). Equation 1 is only a projection based on the k_{H} values obtained using the two drugs. This equation could be used as a template to calculate the k_{H} for drugs that have log P values within the range of 1.76-3.27. The calculated k_{H} value could be used to estimate the amount of drug released at a given time based on the simplified Higuchi equation (Equation 2) shown below. The derived empirical equation could be further validated in the future by encapsulation of various drugs with a range of log P values into the MP-R formulation.

$$y = - 1.6172 x + 6.8 \; 673 , \qquad 1$$

where y is release rate constant (k_{H}) and x is log P

$$Q = {k}_{H} \sqrt{t} , \qquad 2$$

where Q is the amount of drug released at a given time t.

The fabrication of an orally administrable GRDDS has been detailed and its ability to float and be retained under gastric conditions has been proven in vitro using simulated gastric conditions in the above examples. The GRDDS possesses excellent floatability of up to a month with enhanced drug-loading efficiencies, including free drugs or other delivery matrixes, while exhibiting controlled and sustained release of the encapsulant for a week. This work establishes the described hydrogel raft as a platform technology that can be utilized in applications that require the retention of the raft for a prolonged period. It also demonstrates the functionality of the raft to provide sustained release of the encapsulated ingredients, using two model drugs of different solubilities, by controlling their initial burst release and extending their release over a few days. Thus, this work proves the feasibility of developing an ultra-long floating oral delivery system using biopolymers and holds promise that it could further be adopted for other drugs that require long-term administration.

1. A pharmaceutical flotation device suitable for extended release of a pharmaceutical product in a stomach of a subject, the flotation device comprising a polymeric matrix that comprises:
a first polymeric material;
a second polymeric material; and
a crosslinking agent, wherein:
the first polymeric material and the second polymeric material form a hydrogel network; and
the crosslinking agent is configured to generate additional crosslinks in at least the first polymeric material upon exposure to an aqueous solution having a pH of from 1 to 5.

2. The pharmaceutical flotation device according to claim 1, wherein the pharmaceutical flotation device is provided in a first portion shaped as a hollow vessel and one or more second portions shaped as one or both of a cap and a plug, such that a pharmaceutical product can be deposited within the hollow vessel and sealed inside by the one or both of cap and plug.

3. The pharmaceutical flotation device according to claim 1, wherein a pharmaceutical product is encapsulated within the polymeric matrix of the pharmaceutical flotation device.

4. The pharmaceutical flotation device according to claim 1, wherein the polymeric matrix further comprises an oil.

5. The pharmaceutical flotation device according to claim 1, wherein the first polymeric material is selected from one or more of the group consisting of a pectin, and an alginate.

6. The pharmaceutical flotation device according to claim 1, wherein the second polymeric material is selected from one or more of the group consisting of resistant starch, kappa-carrageenan, agarose, iota-carrageenan, cellulose, methylcellulose, ethyl cellulose, and gelatine.

7. The pharmaceutical flotation device according to claim 6,
wherein the second polymeric material is kappa-carrageenan.

8. The pharmaceutical flotation device according to claim 1, wherein the crosslinking agent is a bivalent metal carbonate.

9. The pharmaceutical flotation device according to claim 8, wherein the bivalent metal carbonate is CaCO₃.

10. (canceled)

11. (canceled)

12. The pharmaceutical flotation device according to claim 1, wherein the polymeric matrix comprises:
sodium alginate;
kappa-carageenan; and
CaCO₃, where
the wt:wt ratio of the sodium alginate:kappa-carageenan:CaCO₃ is about 1:1:1.

13. The pharmaceutical flotation device according to claim 2, as dependent upon claim 2, wherein the pharmaceutical flotation device further comprises a pharmaceutical product.

14. (canceled)

15. (canceled)

16. A method of forming a pharmaceutical flotation device according to claim 1, the method comprising the steps of:
(a) providing a solution mixture comprising:
a first polymeric material;
a second polymeric material;
a crosslinking agent; and
water, where the first polymeric material and the second polymeric material interact to form a hydrogel network;
(b) placing the solution mixture into a mould, solidifying the solution mixture and removing the water to provide a pharmaceutical flotation device.

17. The method according to claim 16, wherein step (b) is conducted using at least two moulds, so as to form a first portion shaped as a hollow vessel and one or more second portions shaped as one or both of a cap and a plug, such that a pharmaceutical product can be deposited within the hollow vessel and sealed inside by the one or both of cap an plug.

18. The method according to claim 16, wherein the solution mixture further comprises a pharmaceutical product.

19. The method according to claim 16, wherein the solution mixture further comprises an oil.

20. (canceled)

21. (canceled)

22. (canceled)

23. The method according to claim 16, wherein the crosslinking agent is a bivalent metal carbonate.

24. The method according to claim 23, wherein the bivalent metal carbonate is CaCO₃.

25. (canceled)

26. The method according to claim 25, wherein the maximum concentration of each of the first polymeric material and the second polymeric material in the solution mixture is from 1 to 2 wt %/volume.

27. (canceled)

28. (canceled)

29. The method according to claim 18, as dependent upon claim 18, wherein the pharmaceutical product is in the form of: particles of one or more active pharmaceutical ingredients; particles of one or more active pharmaceutical ingredients and pharmaceutically acceptable excipients; a tableted formulation; a capsule formulation; drug-loaded microparticles; and drug-loaded fibre meshes.

30. (canceled)

31. A pharmaceutical formulation comprising a pharmaceutical flotation device according to claim 1 and a pharmaceutical product.

32. (canceled)
